(19) United States
(12) Patent Application Publication
(10) Pub. No.: US 2022/0130043 A1
(43) Pub. Date: Apr. 28, 2022

(54) METHOD FOR EVALUATING ADHERENT CELL, RECORDING MEDIUM, AND SYSTEM FOR EVALUATING ADHERENT CELL

(71) Applicant: OLYMPUS CORPORATION, Tokyo (JP)

(72) Inventors: Akira MATSUSHITA, Tokyo (JP); Isao SAKANE, Tokyo (JP); Yu HIROSAWA, Tokyo (JP); Takuma DEZAWA, Tokyo (JP)

(73) Assignee: OLYMPUS CORPORATION, Tokyo (JP)

(21) Appl. No.: 17/568,925

(22) Filed: Jan. 5, 2022

Related U.S. Application Data
(63) Continuation of application No. PCT/JP2019/027612, filed on Jul. 11, 2019.

(51) Int. Cl. G06T 7/00; C12M 1/00; C12M 3/00; C12M 1/12

(52) U.S. Cl. CPC G06T 7/0012; C12M 23/22; C12M 23/34; G06T 2207/30024; C12M 25/00; G06T 2207/10024; C12M 23/48

(57) ABSTRACT

A method for evaluating an adherent cell includes: obtaining an image of an adherent cell accommodated within a culture container; and determining whether the adherent cell is adhering to the culture container based on at least a profile shape of a contact portion of the adherent cell that is in contact with the culture container, the profile shape being specified from the image.

### CROSS REFERENCE TO RELATED APPLICATIONS

This application is based upon and claims the benefit of priority to PCT Application No. PCT/JP2019/027612, filed on Jul. 11, 2019, the entire contents of which are incorporated herein by reference.

This is a Continuation Application of PCT Application No. PCT/JP2019/027612, filed Jul. 11, 2019, which was not published under PCT Article 21(2) in English.

### TECHNICAL FIELD

The disclosure herein relates to a method for evaluating an adherent cell, a recording medium, and a system for evaluating an adherent cell.

### BACKGROUND

Cultured cells are roughly divided into adherent cells, which proliferate in a state of adhering to a culture container, and suspension cells, which proliferate in a state of suspending within a container. For evaluation of adherent cells, whether the cells are adhering is highly important information, and for example, a time required before the cells adhere to a culture container, which is obtained based on the information, is known as one effective indicator for evaluating the proliferation potential of the cells.

Techniques pertaining to an adhesion determination for adherent cells are described in, for example, Japanese Laid-open Patent Publication No. 2003-21628. Japanese Laid-open Patent Publication No. 2003-21628 describes a technique for determination about adhesion on the basis of the area of an image of cells projected onto a culture container.

### SUMMARY

A method for evaluating an adherent cell in accordance with an aspect of the present invention includes: obtaining an image of an adherent cell accommodated within a culture container; and determining whether the adherent cell is adhering to the culture container based on at least a profile shape of a contact portion of the adherent cell that is in contact with the culture container, the profile shape being specified from the image.

A recording medium in accordance with an aspect of the present invention is a non-transitory computer-readable recording medium having recorded therein a program for causing a computer to perform a process including: obtaining an image of an adherent cell accommodated within a culture container; and determining whether the adherent cell is adhering to the culture container based on at least a profile shape of a contact portion of the adherent cell that is in contact with the culture container, the profile shape being specified from the image.

A system for evaluating an adherent cell in accordance with an aspect of the present invention includes: an image capturing apparatus configured to capture an image of an adherent cell accommodated within a culture container; and a processor, wherein the processor is configured to obtain the image obtained by the image capturing apparatus, and determine whether the adherent cell is adhering to the culture container based on at least a profile shape of a contact portion of the adherent cell that is in contact with the culture container, the profile shape being specified from the image.

### BRIEF DESCRIPTION OF DRAWINGS

FIG. 1 exemplifies the configuration of an evaluation system 1;

FIG. 2 exemplifies the configuration of an image capturing apparatus 10.

FIG. 3 exemplifies the configuration of a control apparatus 30.

FIG. 4A is a first diagram for illustrating an adhesion process;

FIG. 4B is a second diagram for illustrating an adhesion process;

FIG. 4C is a third diagram for illustrating an adhesion process;

FIG. 4D is a fourth diagram for illustrating an adhesion process;

FIG. 5 is a diagram exemplifying the shape of a contact portion of a cell in a non-adhering state;

FIG. 6A is a diagram exemplifying the shape of a contact portion of a cell in an adhering state;

FIG. 6B is another diagram exemplifying the shape of a contact portion of a cell in an adhering state;

FIG. 6C is still another diagram exemplifying the shape of a contact portion of a cell in an adhering state;

FIG. 7 is yet another diagram exemplifying the shape of a contact portion of a cell in an adhering state;

FIG. 8 is a flowchart for processes in accordance with a first embodiment;

FIG. 9 is a flowchart for an image obtainment process in accordance with a first embodiment;

FIG. 10 is a flowchart for an adhesion determination process in accordance with a first embodiment;

FIG. 11 is an example of a graph indicating temporal changes in a non-adhering cell count and an adhering cell count;

FIG. 12 is a flowchart for an evaluation process in accordance with a first embodiment;

FIG. 13A is a first diagram for illustrating a division process;

FIG. 13B is a second diagram for illustrating a division process;

FIG. 13C is a third diagram for illustrating a division process;

FIG. 13D is a fourth diagram for illustrating a division process;

FIG. 13E is a fifth diagram for illustrating a division process;

FIG. 14 is a flowchart for processes in accordance with a second embodiment;

FIG. 15 is a flowchart for a division determination process in accordance with a second embodiment;

FIG. 16 is an example of a graph indicating temporal changes in a total cell count, a division-started cell count, and a division-completed cell count;

FIG. 17 is a flowchart for an evaluation process in accordance with a second embodiment; and

FIG. 18 is a flowchart for an evaluation process in accordance with a third embodiment.

### DESCRIPTION OF EMBODIMENTS

The relationship between a projection area and an adhering state is not necessarily constant but is inferred to differ according to the cell type. Accordingly, there is demand for a new technique for allowing an adhesion determination for an adherent cell to be accurately performed irrespective of the cell type.

The following describes embodiments of the present invention.

### First Embodiment

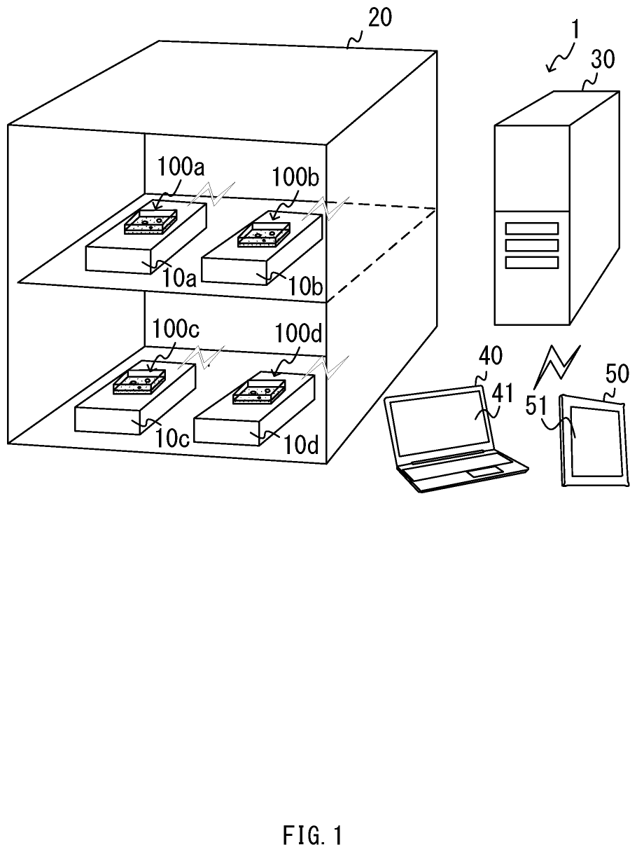

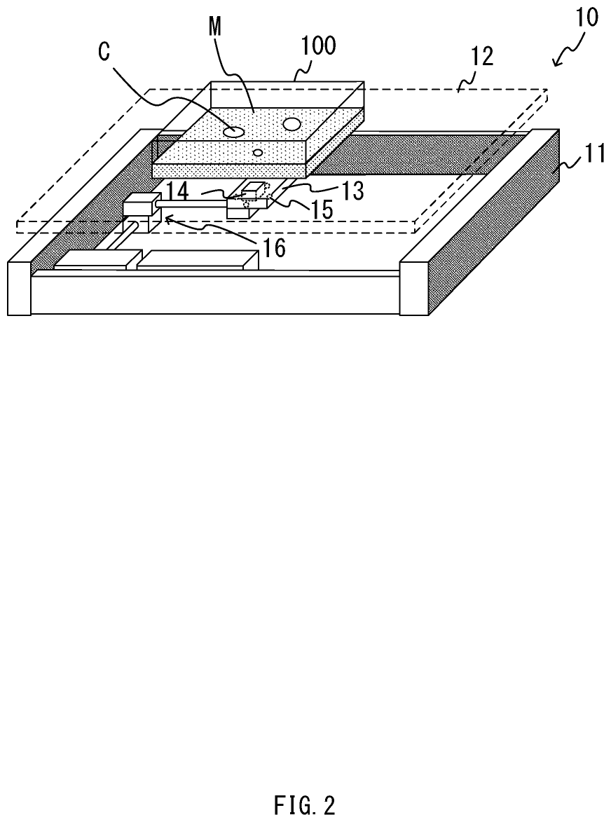

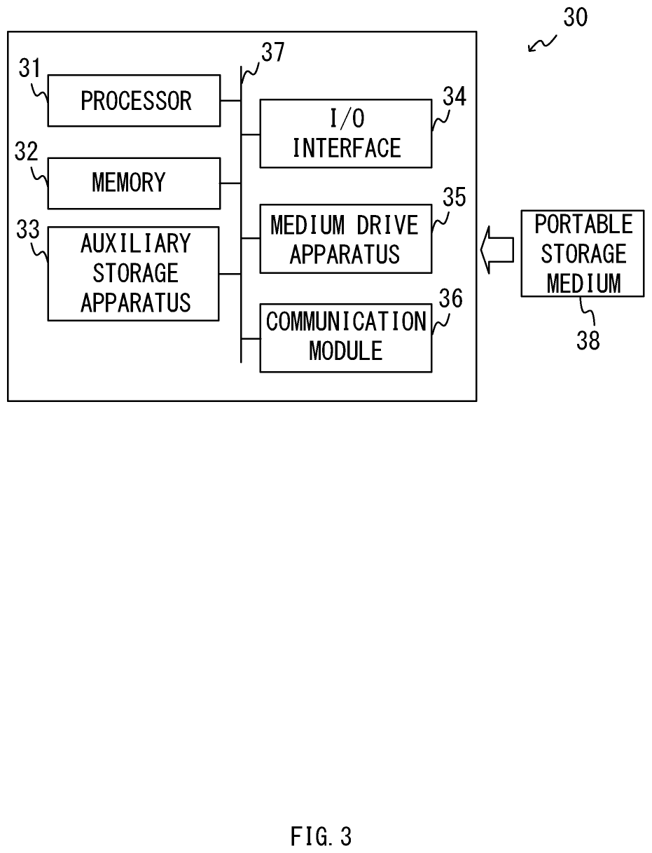

FIG. 1 exemplifies the configuration of an evaluation system 1. FIG. 2 exemplifies the configuration of an image capturing apparatus 10. FIG. 3 exemplifies the configuration of a control apparatus 30. Descriptions are given in the following of the configuration of the evaluation system 1 by referring to FIGS. 1-3.

The system 1 obtains an image of a cultured cell and evaluates the cultured cell on the basis of the image, while culturing the cultured cell in a managed environment within an incubator 20. Note that a cultured cell to be evaluated by the evaluation system 1 is an adherent cell that proliferates in a state of adhering to a culture container.

As depicted in FIG. 1, the evaluation system 1 includes the control apparatus 30 and image capturing apparatuses (image capturing apparatuses 10a, 10b, 10c, and 10d, which are each hereinafter referred to as an image capturing apparatus 10) placed within the incubator 20. In the evaluation system 1, the control apparatus 30 evaluates an adherent cell on the basis of an image obtained by the image capturing apparatus 10. The control apparatus 30 communicates with the image capturing apparatuses 10 and terminal apparatuses (terminal apparatuses 40 and 50). Note that the evaluation system 1 may include the incubator 20 and terminal apparatuses.

As indicated in FIG. 1, culture containers (culture containers 100a, 100b, 100c, and 100d, which are each hereinafter referred to as a culture container 100) are placed on the image capturing apparatuses 10 accommodated within the incubator 20. For example, the culture container 100 may be, but is not particularly limited to, a petri dish, a flask, or a microplate.

At least one adherent cell is accommodated within the culture container 100. More specifically, as depicted in FIG. 2, a medium M and an adherent cell C are accommodated within the culture container 100. For example, the medium M may be, but is not particularly limited to, a solution containing calf blood serum, and the cell C may be, but is not particularly limited to, a chondrocyte. As long as the cell C is an adherent cell, the cell C is not limited to a chondrocyte and may typically be, for example, an epithelial cell, a stromal cell, an established cell line, or a cancer cell. Some hemocyte cells such as dendritic cells require a scaffold during a certain period in a differentiation step. The cells that temporarily require a scaffold may be deemed as adherent cells.

The image capturing apparatus 10 images the adherent cell accommodated within the culture container 100 so as to obtain an image of the adherent cell. The image capturing apparatus 10 includes a wireless communication module (not illustrated). The image capturing apparatus 10 transmits the obtained image of the adherent cell to the control apparatus 30 via wireless communication using the wireless communication module.

More specifically, as depicted in FIG. 2, the image capturing apparatus 10 includes a housing 11 and a stage 12 on which the culture container 100 is placed. The image capturing apparatus 10 also includes an image capturing unit 13 and a moving mechanism 16 for moving the image capturing unit 13, both of which are positioned within the housing 11 and below the stage 12. The image capturing unit 13 is provided with an image capturing element 14, light sources 15, and an optical system (not illustrated).

For example, the image capturing element 14 may be a charge-coupled-device (CCD) image sensor, or a complementary-MOS (CMOS) image sensor. The light sources 15 are, for example, light emitting diodes (LEDs) and illuminate the culture container 100 from below the stage 12. As depicted in FIG. 2, the light sources 15 may be disposed around the image capturing element 14. In the image capturing apparatus 10, light emitted from a light source 15 passes through the bottom surface of the culture container 100, and a portion of light reflected by the upper surface of the culture container 100 passes through the cell C within the culture container 100. The optical system forms an optical image of the cell C on the image capturing element 14 by using the light that has passed through the cell C within the culture container 100.

For example, the moving mechanism 16 may include a drive source such as a motor and move the image capturing unit 13 in directions orthogonal to the optical axis of the optical system (in X and Y directions). The moving mechanism 16 moves the image capturing unit 13 in the X and Y directions, thereby allowing the image capturing apparatus 10 to change the range of image capturing. The moving mechanism 16 may also move the image capturing unit 13 in the direction of the optical axis of the optical system (Z direction). The image capturing apparatus 10 may adjust a focus position by using the moving mechanism 16. Alternatively, the image capturing apparatus 10 may adjust the focus position by moving at least one lens among lenses included in the optical system in the direction of the optical axis.

The control apparatus 30 is a computer that controls the evaluation system 1. As depicted in FIG. 3, the control apparatus 30 includes a processor 31, a memory 32, an auxiliary storage apparatus 33, an I/O interface 34, a medium drive apparatus 35 for driving a portable storage medium 38, a communication module 36, and a bus 37. The auxiliary storage apparatus 33 and the portable storage medium 38 are each an example of a non-transitory computer-readable recording medium storing a program.

For example, the processor 31 may be any processing circuit that includes a central processing unit (CPU) and/or a graphics processing unit (GPU). The processor 31 performs programmed processing, e.g., various types of processing described hereinafter, by loading a program stored in the auxiliary storage apparatus 33 or the portable storage medium 38 into the memory 32 and then executing the loaded program.

For example, the memory 32 may be any semiconductor memory such as a random access memory (RAM). In program execution, the memory 32 functions as a work memory for storing a program or data stored in the auxiliary storage apparatus 33 or the portable storage medium 38. For example, the auxiliary storage apparatus 33 may be a nonvolatile memory such as a hard disk or a flash memory. The auxiliary storage apparatus 33 is used mainly to store various data and programs.

The medium drive apparatus 35 accommodates the portable storage medium 38, e.g., an optical disc or Compact Flash®. The medium drive apparatus 35 can output data stored in the memory 32 or the auxiliary storage apparatus 33 to the portable storage medium 38 and read a program, data, and the like from the portable storage medium 38. The portable storage medium 38 may be any storage medium that can be carried. For example, the portable storage medium 38 may include an SD card, a universal-serial-bus (USB) flash memory, a compact disc (CD), and a digital versatile disc (DVD).

For example, the input/output (I/O) interface 34 may be a universal-serial-bus (USB) interface circuit or a High-Definition Multimedia Interface (HDMI)® circuit. The I/O interface 34 has, for example, an input apparatus (e.g., a keyboard, a mouse) (not illustrated) and an output apparatus (e.g., a display, a printer) (not illustrated) connected thereto.

For example, the communication module 36 may be a wireless communication module. The standard for wireless communication is not particularly limited and may be, for example, Bluetooth®, Low Energy (hereinafter, “BLE”), or Wi-Fi®. The communication module 36 may be a wire communication module. The control apparatus 30 communicates with the image capturing apparatuses 10 and the terminal apparatuses by using the communication module 36. The bus 37 connects the processor 30, the memory 32, the auxiliary storage apparatus 33, and the like to each other in a manner such that data can be communicated therebetween.

The control apparatus 30 and the image capturing apparatuses 10 may be formed as a single body. When the control apparatus 30 and the image capturing apparatuses 10 are formed as a single body, a wired system is preferably used for communication between the control apparatus 30 and the image capturing apparatuses 10.

The configuration depicted in FIG. 3 is an example of the hardware configuration of the control apparatus 30. The control apparatus 30 is not limited to this configuration. The control apparatus 30 may be a general-purpose or special-purpose apparatus. For example, the control apparatus 30 may include a specifically designed electric circuit, e.g., an application specific integrated circuit (ASIC). The control apparatus 30 may be configured using a field-programmable gate array (FPGA).

The terminal apparatus 40 is a notebook computer. The terminal apparatus 50 is a tablet computer. The terminal apparatuses each include a display (display 41, display 51) constituting a display unit and causes the display to display, for example, an evaluation result and an image of an adherent cell received from the control apparatus 30. As long as the terminal apparatus includes a display unit, the terminal apparatus may be, for example, a desktop computer or a smartphone. By accessing the evaluation system 1 with the terminal apparatus, the user can observe an adherent cell and check an evaluation result.

In the evaluation system 1 configured as described above, when evaluating adherent cells accommodated within the culture container 100, an adhesion determination for cells C is performed on the basis of an image of the cells C obtained by the image capturing apparatus 10.

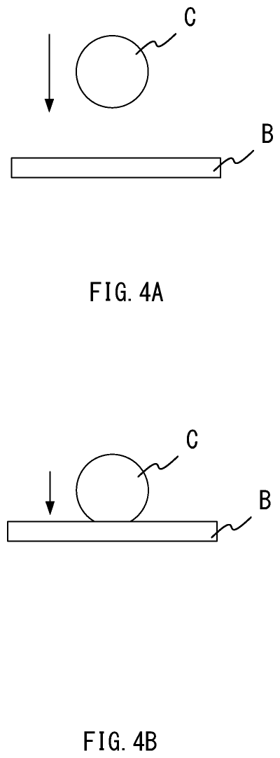

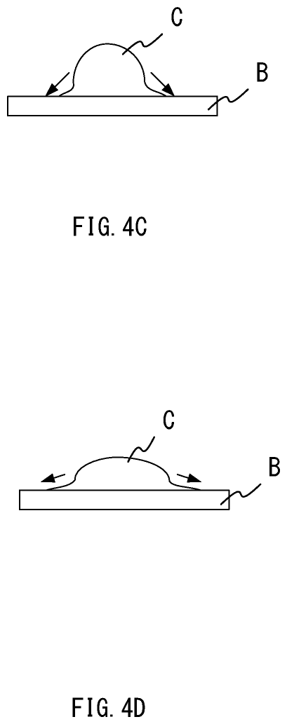

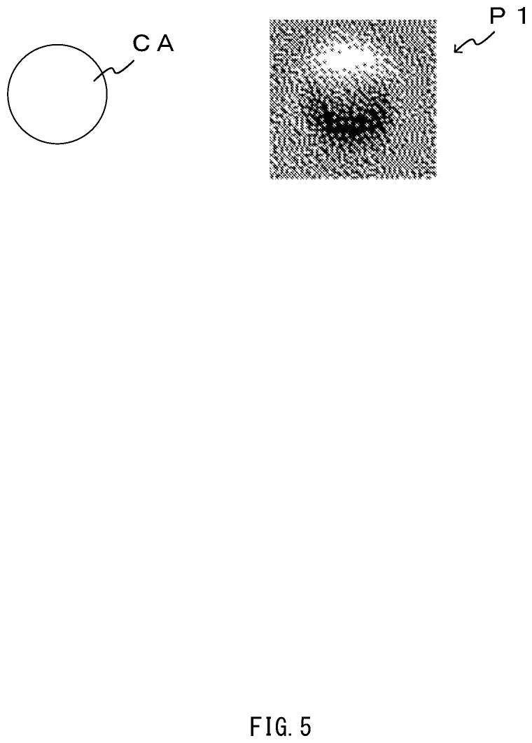

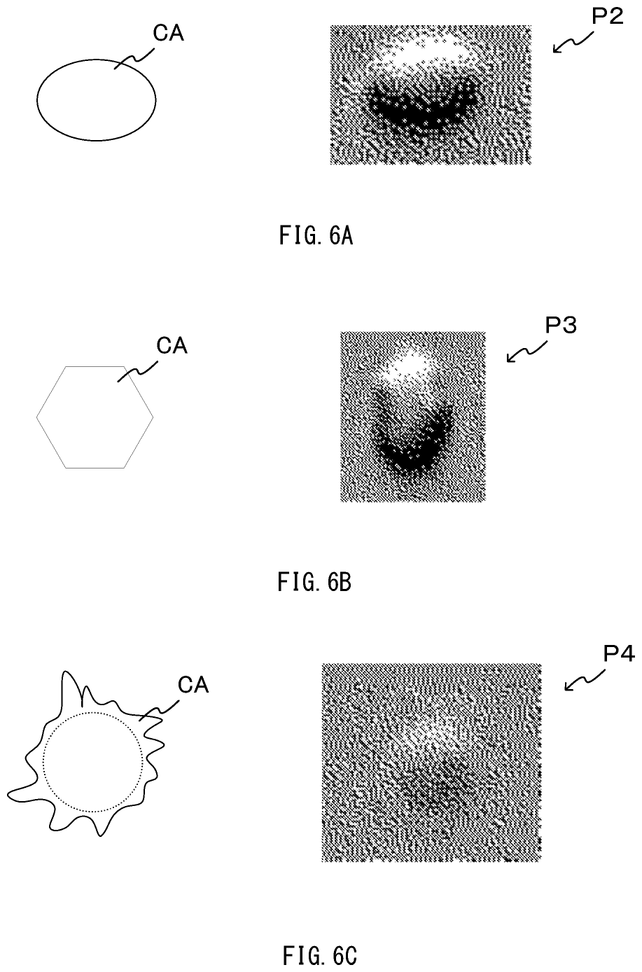

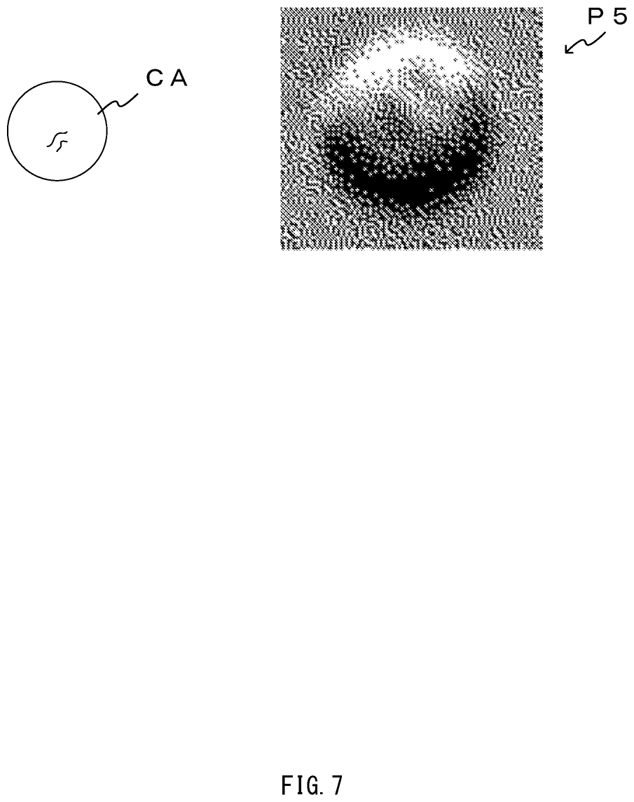

FIGS. 4A-4D are diagrams for illustrating an adhesion process. FIG. 5 is a diagram exemplifying the shape of a contact portion of a cell in a non-adhering state. FIGS. 6A-6C and 7 are diagrams exemplifying the shape of a contact portion of a cell in an adhering state. Images P1-P5 in FIGS. 5, 6A-6C, and 7 are pictures indicating various cell states. By referring to FIGS. 4A-4D, 5, 6A-6C, and 7, the following describes a change in the shape of a cell through a process from seeding to adhesion (hereinafter simply referred to as an “adhesion process”), more specifically a change in the profile shape of the cell, as well as a method for an adhesion determination implemented by the evaluation system 1.

When a cell C is seeded within a culture container 100 in which a medium M is accommodated, the cell C will start to settle out toward a bottom surface B in the culture container 100, as depicted in FIG. 4A. While the cell C is settling, the shape of the cell C, more specifically the profile shape thereof, may be deemed as a spherical shape.

As the settling progresses, the cell C will come into contact with the bottom surface B. When the cell C is in contact with the bottom surface B, a portion of the cell C in contact with the bottom surface B is flattened as indicated in FIG. 4B. Thus, the profile shape of the cell C can be deemed as the shape of a spherical segment. In this case, when seen from the bottom-surface-B side, the profile shape of a contact portion CA of the cell C in contact with the culture container 100 (hereinafter simply referred to as a “contact portion”) is a circular shape, as indicated in FIG. 5. This is because the cell C has not adhered to the culture container 100 yet, i.e., is in a so-called non-adhering state. During the non-adhering state, an unbalanced stress that could break the circular shape of the contact portion CA does not act between the cell C and the culture container 100 (bottom surface B).

Afterward, the cell C adheres to the bottom surface B (culture container 100). As the adhesion progresses, the cell C spreads over the bottom surface B and collapses, as depicted in FIGS. 4C and 4D. During a state in which the cell C adheres to the culture container 100, i.e., a so-called adhering state, an anisotropic unbalanced stress acts between the cell C and the bottom surface B. Thus, the profile shape of the contact portion CA is, in most cases, a non-circular shape, unlike during the non-adhering state. In particular, the profile shape of the contact portion CA is, for example, an oval shape such as that depicted in FIG. 6A, a polygonal shape such as that depicted in FIG. 6B, or a shape with pseudopodia such as that depicted in FIG. 6C. Note that “pseudopodia (pseudopod)” refers to a structure of protrusions of a cell.

As described above, the profile shape of the contact portion CA between the adherent cell and the culture container 100 can be deemed as a circular shape during the non-adhering state and can be deemed as a shape different from a circular shape during the adhering state. This change unavoidably occurs in accordance with adhesion and thus arises irrespective of the cell type. By taking advantage of this change, the evaluation system 1 performs the adhesion determination on the basis of the profile shape of the contact portion CA. Accordingly, the evaluation system 1 can accurately perform the adhesion determination for an adherent cell irrespective of the cell type.

Although it is rare, the contact portion CA may assume a circular profile shape by chance in association with a stress balance even during the adhering state. In such a case, it may be determined whether the cell is in the adhering state or the non-adhering state on the basis of the presence/absence of irregularities such as wrinkles on a face of the adherent cell in contact with the container. During the adhering state, in comparison with the non-adhering state, a large stress arises between the adherent cell and the culture container 100, so wrinkles tend to be formed on the contact portion CA, as depicted in, for example, FIG. 7. Thus, when there are irregularities on a face of the adherent cell, such as on the contact portion CA, it may be determined that the adherent cell is in the adhering state. Considering the presence/absence of irregularities in addition to the shape of the contact portion CA allows the adhesion determination for the adherent cell to be performed more accurately. The method for determining the presence/absence of irregularities such as wrinkles is not particularly limited and may be a method involving using blob analysis or a method involving detecting wrinkles by learning the same through machine learning.

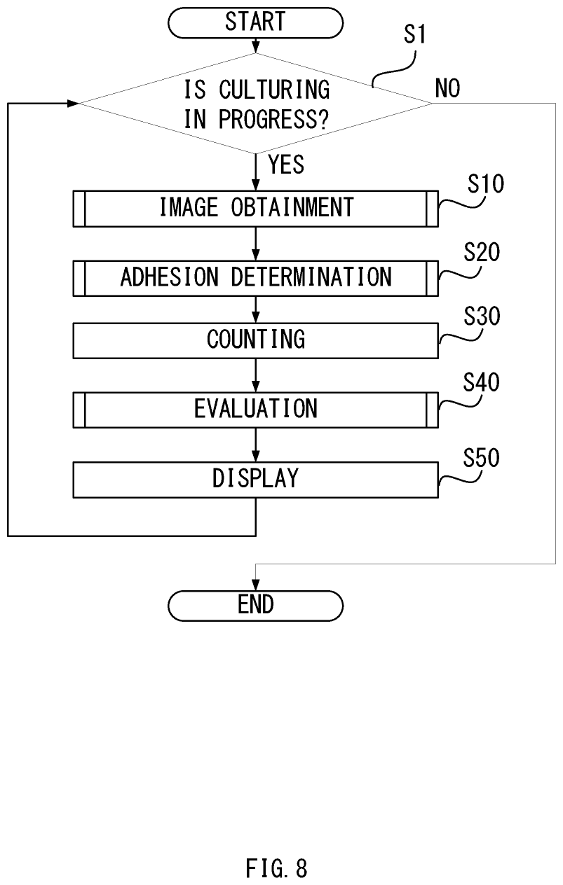

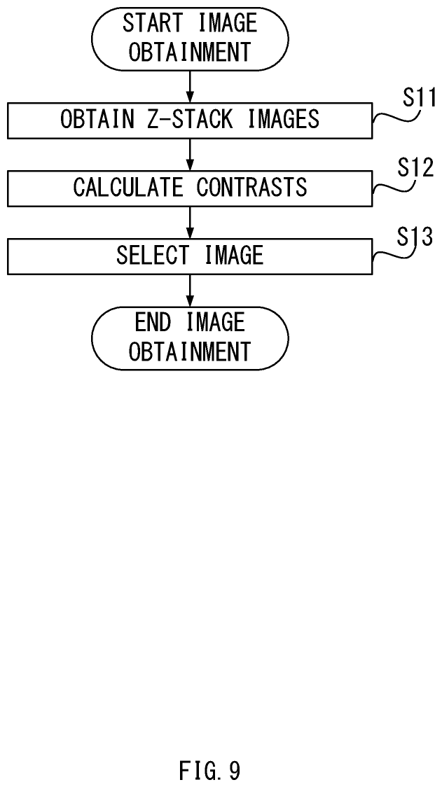

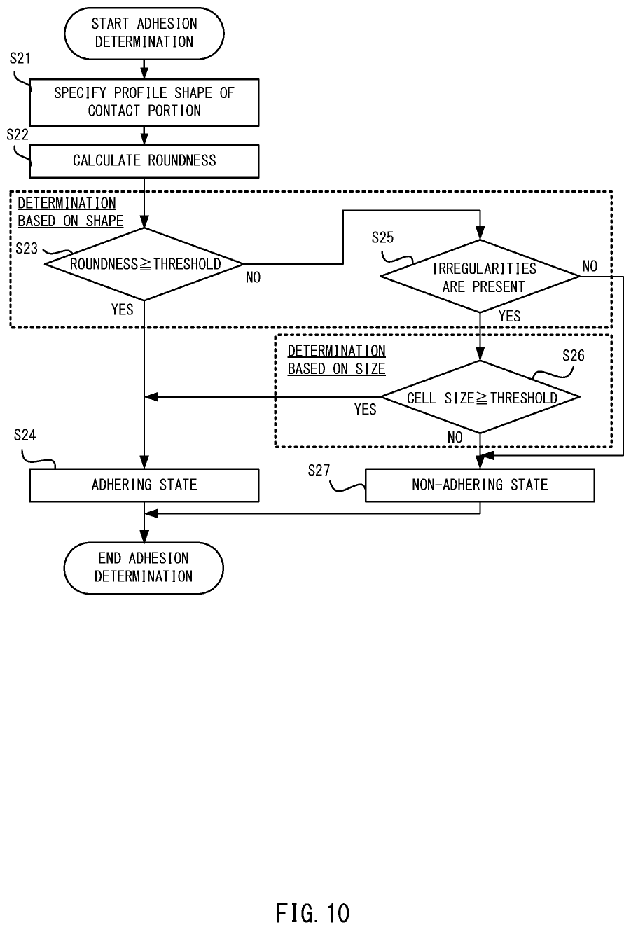

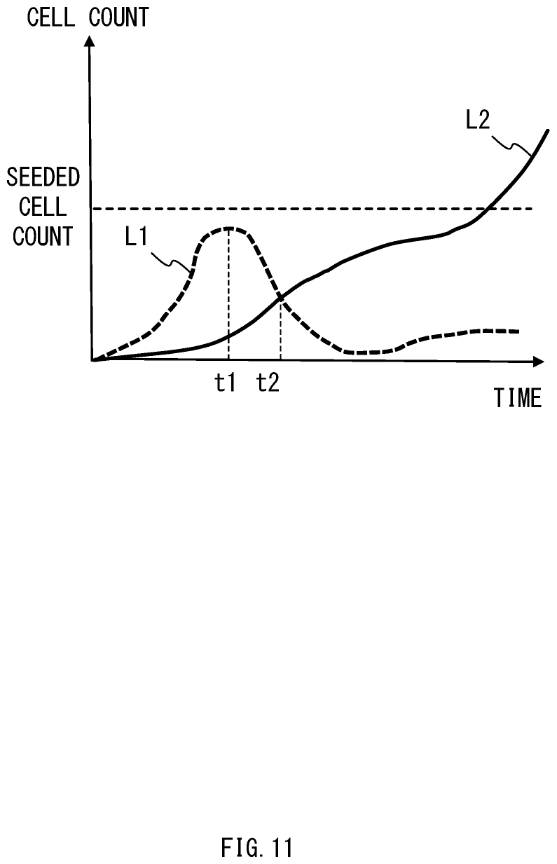

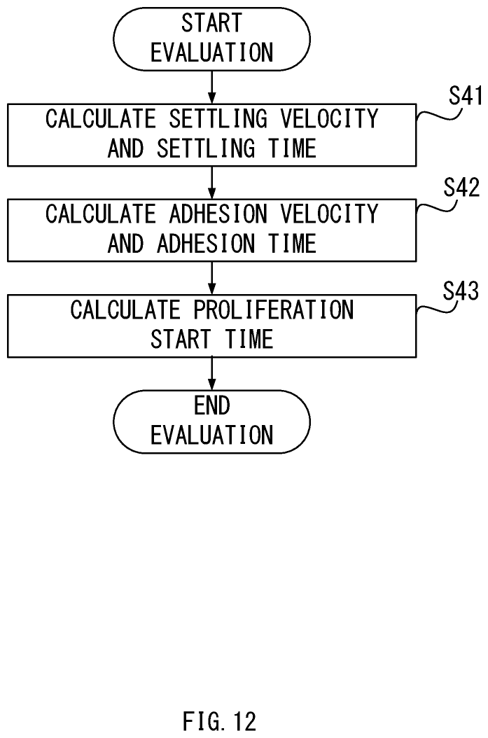

FIG. 8 is a flowchart for processes in accordance with the present embodiment that are performed by the evaluation system 1. FIG. 9 is a flowchart for an image obtainment process in accordance with the present embodiment. FIG. 10 is a flowchart for an adhesion determination process in accordance with the present embodiment. FIG. 11 is an example of a graph indicating temporal changes in a non-adhering cell count and an adhering cell count. FIG. 12 is a flowchart for an evaluation process in accordance with the present embodiment. By referring to FIGS. 8-12, the following specifically describes a method for evaluating an adherent cell that is implemented by the evaluation system 1.

As depicted in FIG. 8, the method for evaluating an adherent cell that is implemented by the evaluation system 1 includes a culturing progression determination process (step S1), an image obtainment process (step S10), an adhesion determination process (step S20), a counting process (step S30), an evaluation process (step S40), and a display process (step S50). In this example, time-lapse image capturing is performed.

First, the evaluation system determines whether cell culturing is in progress (step S1). When culturing is in progress, the processes of step S10 and the following steps are repeatedly performed. When culturing is not in progress, the processes in FIG. 8 end. In the image obtainment process in step S10, the evaluation system 1 obtains images to be used in the adhesion determination process in step S20. In particular, the image capturing apparatus 10 obtains images of one or more cells C accommodated within the culture container 100 and transmits the obtained images to the control apparatus 30. Hence, the control apparatus 30 obtains the images of one or more cells C.

In the adhesion determination in step S20, the shape of a contact portion CA is used as described above. In step S10, accordingly, for example, the evaluation system 1 may use an autofocus function of the image capturing apparatus 10 so as to set a focus position on the bottom surface in the culture container 100 to which the cell C could adhere, and then obtain images. In this way, images with which the shape of the contact portion CA can be easily specified can be obtained.

In step S10, the evaluation system 1 may perform the image obtainment process indicated in FIG. 9. Also when performing the image obtainment process indicated in FIG. 9, the evaluation system 1 can obtain images with which the shape of the contact portion CA can be easily specified.

In the image obtainment process indicated in FIG. 9, the evaluation system 1 first obtains Z-stack images (step S11). In step S11, the image capturing apparatus 10 moves the focus position in increments of certain distances in the direction of an optical axis so as to obtain an image at each position. That is, a plurality of images are obtained for different focus positions. Then, the image capturing apparatus 10 transmits the plurality of obtained images to the control apparatus 30.

Upon receipt of the plurality of images obtained for different focus positions, the control apparatus 30 calculates the contrast of each of the plurality of images (step S12). The method for calculating the contrast of an image is not particularly limited and may be, for example, Brenner Gradient. In Brenner Gradient, the squares of the differences in pixel value between neighboring pixels are integrated within a certain region. The certain region for integration may be the entirety of an image or a region of interest in the image.

Subsequently, on the basis of the contrasts of the plurality of images calculated in step S12, the control apparatus 30 selects a selection image to be used in the adhesion determination in step S20 (step S13). In step S13, for example, the control apparatus 30 may select the image with the highest contrast as the selection image.

Upon completion of the image obtainment process in step S10, the evaluation system 1 performs the adhesion determination process (step S20). In step S20, the evaluation system 1 determines whether the cell C is adhering to the culture container 100 on the basis of the profile shape of the contact portion CA, which is specified from the images obtained in step S10. When the image obtainment process depicted in FIG. 9 is performed in step S10, in step S20, the adhesion determination process will be performed on the basis of the selection image. In step S20, the adhesion determination process is performed for each of the plurality of cells C in the image so as to determine the state of the plurality of cells C (adhering state, non-adhering state).

The image obtainment in step S10 is performed as appropriate during cell culturing. In time-lapse image capturing, for example, image obtainment may be performed, as described above, at regular intervals during cell culturing and may be additionally preformed at irregular intervals during the cell culturing. Meanwhile, every time an image is obtained, an analysis process that includes the adhesion determination and the evaluation performed in step S20 and the following steps (described hereinafter) may be carried out in temporally parallel to cell culturing. A series of images obtained during culturing may be collectively analyzed after the culturing ends.

The adhesion determination process in step S20 may be performed using machine learning, in particular deep learning. For example, a learned model that has learned the profile shapes of the contact portions CA of cells in the adhering state and the profile shapes of the contact portions CA of cells in the non-adhering state may be built, and the images obtained in step S10 may be input to the learned model so as to perform the adhesion determination. Alternatively, a learned model that has learned the shapes of pseudopodia may be built, and the images obtained in step S10 may be input to the learned model so as to perform the adhesion determination. Learning the profile shapes of contact portions CA allows the adhesion determination for an adherent cell to be accurately performed irrespective of the cell type. In addition to the profile shapes of contact portions CA, the learned model may learn, for example, irregularities on the surfaces of cells in the adhering state and irregularities on the surfaces of cells in the non-adhering state. In this way, a more accurate adhesion determination can be performed.

A teacher data set for creating a learned model is formed from cell images obtained for model creation and annotation data provided by a model creator or person with specialized knowledge determining the profile shapes of cells in the cell images or determining the presence/absence of pseudopodia in the cell images. The learned model may be refined as appropriate. In particular, for example, while the system 1 is used, an operator may perform the adhesion determination through a visual check and additionally create annotation data so as to refine the learned model, and refine the learned model by additionally performing learning by using teacher data including the additionally created annotation data.

In step S20, the evaluation system 1 may perform the adhesion determination process depicted in FIG. 10. In the adhesion determination process depicted in FIG. 10, the evaluation system 1 first specifies the profile shape of the contact portion CA (step S21). In step S21, the control apparatus 30 specifies the profile shape of the contact portion CA on the basis of the images obtained in step S10. More specifically, the control apparatus 30 may specify the profile shape of the contact portion CA by performing, for example, a profile extraction process for the images obtained in step S10.

Each of the images used in the adhesion determination process indicates a plurality of cells. The adhesion determination process can be performed concurrently for a plurality of cells by processing the images once.

Next, the control apparatus 30 determines whether the cell C is adhering to the culture container 100 on the basis of a deviation of the profile shape of the contact portion CA specified in step S21 from a standard shape (steps S22-S24). When the profile shape of the contact portion CA largely deviates from the standard shape, it can be decided that a stress that arises when the cell C is adhering to the culture container 100 has been generated, thereby determining that the cell C is adhering to the culture container 100. In this example, the standard shape is a circular shape.

In particular, the control apparatus 30 first calculates a roundness on the basis of the profile shape specified in step S21 (step S22). The roundness is defined as the degree of a deviation of a circular shape from a geometrically correct circle. In step S22, the control apparatus 30 calculates, as a roundness, the difference in radius between two concentric circles sandwiching the profile shape specified in step S21, with the distance between the two concentric circles minimized.

Upon the roundness being calculated, the control apparatus 30 determines whether the roundness calculated in step S22 is equal to or greater than a threshold (step S23). For example, the threshold used in step S23 may be set in advance in consideration of a cell size. A threshold may be set for each cell size, and in this case, the control apparatus 30 may specify, in step S22, the roundness and the cell size (e.g., maximum diameter) and use a threshold that corresponds to the cell size specified in step S23. Alternatively, in step S22, the control apparatus 30 may specify the roundness and the cell size (e.g., maximum diameter) and calculate the value of the roundness divided by the cell size. In this case, the control apparatus 30 may determine in step S23 whether the value of the roundness divided by the cell size is equal to or greater than a threshold.

When it is determined in step S23 that the roundness is equal to or greater than the threshold (YES in step S23), the control apparatus 30 determines that the cell C is in the adhering state (step S24). That is, the control apparatus 30 determines that the cell C is adhering to the culture container 100. This is because, when the roundness is equal to or greater than the threshold, it can be considered that the shape of the contact portion CA has been sufficiently changed from a circular shape and thus an occurrence of a stress generated when the cell C is adhering has been observed.

When it is determined in step S23 that the roundness is less than the threshold (NO in step S23), the control apparatus 30 also determines, on the basis of the presence/absence of irregularities on the surface of the cell C, whether the cell C is adhering to the culture container 100 (steps S25 and S26). This is because, when there are irregularities on the surface, it can be considered that a stress has been generated due to adhesion of the cell C. In this example, irregularities on the surface are wrinkles on the contact portion CA. The portions with wrinkles exhibit a lower contrast than the peripheral portion of the cell and are thus considered to have a different height from the peripheral portion. The peripheral portion of the cell is separated from the bottom surface in the container, so it is considered that the portions with wrinkles are adhering to the container.

In particular, the control apparatus 30 first determines, on the basis of the images obtained in step S10, whether there are irregularities on the surface of the cell C (step S25). In this case, for example, the control apparatus 30 may determine that there are irregularities when the contact portion CA is wrinkled, and may determine that there are no irregularities when the contact portion CA is not wrinkled.

When it is determined in step S25 that there are no irregularities (NO in step S25), the control apparatus 30 determines that the cell C is in the non-adhering state (step S27). When it is determined in step S25 that there are irregularities (YES in step S25), the control apparatus 30 determines whether the size of the cell C, which has a contact portion CA, is equal to or greater than a threshold (step S26).

In steps S23 and S25, it is determined whether the cell is adhering on the basis of the shape of the cell (including the profile shape and the surface shape), while in step S26, it is determined whether the cell is adhering on the basis of the cell size. This is intended to ensure a high accuracy in the determination by using the cell size as additional information when the shape of the cell is one that may be assumed during both the adhering state and the non-adhering state. The cell size is associated with the life and death of the cell, so step S26 is substantially a step for determining whether the cell is alive. It can be decided that the cell is a living cell when the cell size is equal to or greater than a threshold, and that the cell is a dead cell when the cell size is less than the threshold. By performing the process of step S26, even when it cannot be specified from the shape of the cell whether the cell is in the adhering state or the non-adhering state, it can be specified whether the cell is in the adhering state or the non-adhering state in consideration of whether the cell is alive.

The size of the cell C may be inferred from the size of the contact portion CA, or the size of the contact portion CA may be deemed as the size of the cell C. For example, the threshold used in step S26 may be set in advance in consideration of the size of a dead cell. The cell size varies according to the cell type, so a specific threshold may be prepared in advance for each cell type.

When it is determined in step S26 that the size of the cell C is equal to or greater than the threshold, the control apparatus 30 determines that the cell C is in the adhering state (step S24). That is, the control apparatus 30 determines that the cell C is adhering to the culture container 100. When it is determined in step S26 that the size of the cell C is less than the threshold, the control apparatus 30 determines that the cell C is dead and thus in the non-adhering state (step S27). That is, the control apparatus 30 determines that the cell C is not adhering to the culture container 100. The surface of the cell C is wrinkled when the cell C dies. Accordingly, by determining in step S26 whether the size of the cell C is equal to or greater than the threshold, wrinkles on a cell that has been dead and thus has a reduced size can be prevented from being mistakenly identified as wrinkles that are formed upon adhesion. The non-adhering state and the dead state may be managed while distinguishing therebetween. Note that formation of wrinkles associated with cell death may not result from a stress. In particular, the cause of formation of wrinkles may be formation of a hole in the cell membrane or an inability to maintain the cytoskeleton.

When the adhesion determination process in step S20 ends, the system 1 performs the counting process (step S30). In step S30, the control apparatus 30 obtains an adhering-cell count and a non-adhering cell count from the images. A method such as machine learning may be used for the cell counting method.

In time-lapse image capturing, the control apparatus 30 may obtain information depicted in FIG. 11 by repeatedly performing the processes of steps S10-S30. A graph is used herein for the information. In FIG. 11, the horizontal axis indicates the elapsed time since seeding, and the vertical axis indicates the number of cells specified from the images (the cell counts obtained in step S30). A dashed line L1 indicates a temporal change in the non-adhering cell count specified from the images, and a solid line L2 indicates a temporal change in the adhering cell count specified from the images. The following describes typical changes in cell counts by referring to FIG. 11.

First, cells that have been floating within a medium after seeding gradually settle out and reach the bottom surface in the culture container 100. Thus, the number of non-adhering cells remarkably increases for a while after the seeding (see line L1). Meanwhile, the number of adhering cells increases when non-adhering cells adhere to the bottom after reaching the bottom. Thus, the number of adhering cells increases after the number of non-adhering cells increases (see line L2).

The number of non-adhering cells reaches a peak at a time t1 at which the increase in the number of non-adhering cells caused by the settling stops, and then the number of non-adhering cells decreases (see line L1). Meanwhile, the number of adhering cells continues to increase (see line L2) with the progress of adhesion of non-adhering cells. The number of adhering cells reaches the number of non-adhering cells at a time t2 and eventually surpasses the number of non-adhering cells (see lines L1 and L2).

Afterward, the number of non-adhering cells starts to increase again (see line L1). This is because the adhering cells, when dividing, transition from the adhering state to the non-adhering state. The adhering cells divide after transitioning to the non-adhering state and then adhere to the culture container 100 again. Meanwhile, the adhering cells, when starting to be divide, slowly increase in number (see line L2) because of the cell division with the transition to the non-adhering state associated with the division. As a result, the adhering cell count surpasses the seeded cell count. When the total cell count increases with the progress of the division, the increase in the cell count associated with the division is accelerated (see line L2). Hence, the rate of increase in the adhering cell count increases.

Next, the evaluation system 1 performs the evaluation process (step S40). In step S40, the evaluation system 1 evaluates various characteristics of the cell C on the basis of an adhesion determination result obtained in step S20. In particular, in step S40, the evaluation system 1 performs the evaluation process depicted in FIG. 12.

In the evaluation process depicted in FIG. 12, the evaluation system 1 first calculates a settling velocity and a settling time (step S41). In step S41, the control apparatus 30 repeatedly calculates the slope of the line L1 until the time t1 at which the number of non-adhering cells reaches a peak, and specifies the calculated slopes as settling velocities at individual times. In addition, the time t1 at which the number of non-adhering cells reaches a peak is calculated as a settling time. For example, the settling time may be used to specify a period in which cell counting can be correctly performed. During a floating state directly after seeding, cells cannot be imaged by performing image capturing from the bottom surface of the container. Accordingly, by deciding that cell counting can be correctly performed after the settling time elapses, the control apparatus 30 may perform cell counting after the settling time elapses.

After calculating the settling velocity and the settling time, the evaluation system 1 calculates an adhesion velocity and an adhesion time (step S42). In step S42, the control apparatus 30 repeatedly calculates the slope of the line L2 until the time t2 at which the number of adhering cells reaches the number of non-adhering cells, and specifies the calculated slopes as adhesion velocities at individual times. In addition, the time t2 at which the number of adhering cells reaches the number of non-adhering cells is calculated as an adhesion time. For example, the adhesion time may be used to estimate the division potential (proliferation potential) of the cells.

After calculating the adhesion velocity and the adhesion time, the evaluation system 1 finally calculates a proliferation start time (step S43) and ends the evaluation process. In step S43, the control apparatus 30 repeatedly decides whether the number of adhering cells has reached a certain threshold, and specifies, as the proliferation start time, the time at which the threshold is reached. For example, the proliferation start time may be used to distinguish between a pre-proliferation period and a proliferation period. The control apparatus 30 may specify the period from the seeding to the proliferation start time as the pre-proliferation period and specify the period from the start of proliferation as the proliferation period.

Upon finishing the evaluation process, the evaluation system 1, at a request from a terminal apparatus, outputs various information to the terminal apparatus and causes the display unit of the terminal apparatus to display the information (step S50). In this case, the control apparatus 30 may cause the display unit of the terminal apparatus to display the adhesion determination result obtained in step S20 in, for example, the graph form depicted in FIG. 11, or may cause the display unit of the terminal apparatus to display an evaluation result obtained in step S40 in, for example, a table form.

The evaluation system 1 configured as described above determines whether an adherent cell is adhering on the basis of the profile shape of a contact portion. The profile shape of the contact portion is not so dependent on the cell type in comparison with the cell size, so that the evaluation system 1 can accurately perform the adhesion determination for an adherent cell irrespective of the cell type.

The evaluation system 1 can use, in addition to the profile shape of a contact portion, irregularities on the surface of a cell for the adhesion determination. Thus, the possibility of an adhering cell being mistakenly recognized as a non-adhering cell can be further reduced. The adhesion determination using irregularities may be performed in combination with a cell size. The combination with the cell size allows wrinkles on a dead cell to be prevented from being mistakenly recognized as irregularities on an adhering cell, so that the adhesion determination can be performed more accurately.

The evaluation system 1 can evaluate an adhering cell by using an adhesion determination result. In particular, the evaluation system 1 can accurately perform the adhesion determination and thus can make an evaluation on the basis of correct information. As a result, the reliability of evaluation can be enhanced in comparison with the prior art.

Specifically, the evaluation system 1 can correctly count the number of non-adhering cells through the adhesion determination and thus can correctly calculate the settling velocity and the settling time. Calculating the settling velocity and the settling time allows for specification of a state in which all of the seeded cells have reached the bottom. Thus, the period in which cell counting can be correctly performed can be specified, thereby allowing correct cell counting to be started as early as possible. The settling velocity and the settling time are dependent on, for example, the depth of the medium M, the viscosity of the medium, the specific weight of the medium, the specific weight of the cells, the shape of the cells, the state of agitation of the cells, and the task of seeding the cells. Accordingly, the settling velocity and the settling time can be used as information for deciding whether the depth of the medium M, the viscosity of the medium, the specific weight of the medium, the specific weight of the cells, the shape of the cells, the state of agitation of the cells, or the task of seeding the cells was appropriate.

The evaluation system 1 can correctly count the number of adhering cells and the number of non-adhering cells through the adhesion determination and thus can correctly calculate the adhesion velocity and the adhesion time. If a cell C is damaged, e.g., if the collagen on the surface of the cell C is excessively lost through trypsinization, the adhesion velocity will decrease, and the adhesion time will be extended. Thus, the calculated adhesion velocity and adhesion time can be used as information for deciding the degree of damage on the cell C. Accordingly, the evaluation system 1 can estimate the division potential of the cell on the basis of the calculated adhesion velocity and adhesion time.

The evaluation system 1 can correctly count the number of adhering cells through the adhesion determination and thus can correctly calculate a proliferation start time. Thus, the pre-proliferation period can be distinguished from the proliferation period so that various information can be associated with the pre-proliferation period and the proliferation period and managed.

In the example indicated for the adhesion determination process depicted in FIG. 10, a deviation of the profile shape of a contact portion CA from a standard shape is evaluated using a roundness. However, a deviation from the standard shape may be evaluated using an evaluation value other than a roundness. For example, the control apparatus 30 may evaluate a deviation by using a circularity instead of a roundness. The circularity of a contact portion CA can be calculated by (circumference of a circle having an equal area to the contact portion CA)/(length of the outer edge of the contact portion CA), and a circularity that is closer to 1 indicates that the contact portion CA is closer to a circle. The evaluation system 1 can also achieve similar effects when a deviation is calculated on the basis of a circularity.

### Second Embodiment

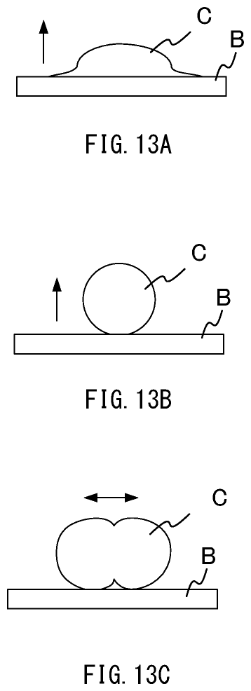

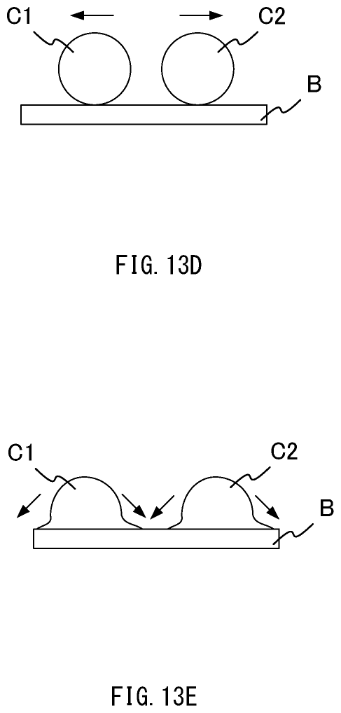

FIGS. 13A-13E are diagrams for illustrating a division process. The evaluation system in accordance with the present embodiment (hereinafter simply referred to as the “evaluation system”) is different from the evaluation system 1 in accordance with the first embodiment in that the former evaluation system performs a division determination in addition to the adhesion determination. By referring to FIGS. 13A-13E, the following describes a change in the shape of a cell in a division process and also describes a method for a division determination in the evaluation system.

A cell C in an adhering state proliferates through cell division. When the division process starts, the cell C changes from a collapsed shape such as that depicted in FIG. 13A to a shape like a sphere such as that depicted in FIG. 13B. This change is opposite to the change from the shape in FIG. 4B to the shape in FIG. 4C in the adhesion process and is thus a change from the adhering state to the non-adhering state. Accordingly, when seen from the bottom-surface-B side, the contact portion CA of the cell C changes from any of the shapes depicted in FIGS. 6A-6C and 7 to the shape depicted in FIG. 5.

Afterward, the cell C starts to divide as depicted in FIG. 13C and changes into a pair of cells such as those depicted in FIG. 13D (cells C1 and C2). In FIG. 13D, the pair of cells remain in the non-adhering state, and thus the respective contact portions CA of the pair of cells maintain the shape depicted in FIG. 5.

However, a change from the adhering state to the non-adhering state also occurs when a cell dies. When a cell dies, the division depicted in FIG. 13C does not occur, and irregularities are formed in the shape depicted in FIG. 5, thereby decreasing the area of the cell, so that the state of cell death can be easily distinguished from the state of dividing.

Finally, adhesion starts again, and, as depicted in FIG. 13E, the pair of cells each spread over the bottom surface B and collapse, thereby adhering to the bottom surface B (culture container 100). Accordingly, the shape of the contact portion CA of each cell of the pair of cells changes to any of the shapes depicted in FIGS. 6A-6C and 7.

As described above, it can be inferred that an adherent cell has been dividing when the cell changes from the adhering state to the non-adhering state. Thus, the evaluation system performs the division determination on the basis of a determination result from the adhesion determination process. Accordingly, the evaluation system can accurately perform, as with the adhesion determination, the division determination for an adherent cell irrespective of the cell type.

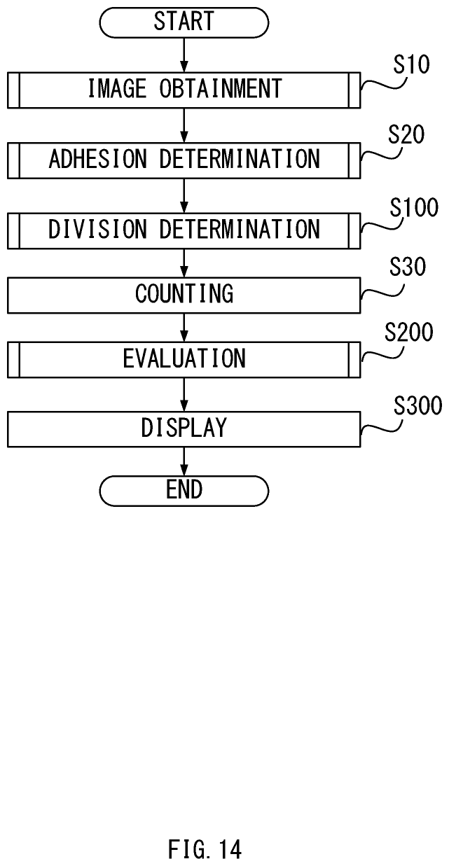

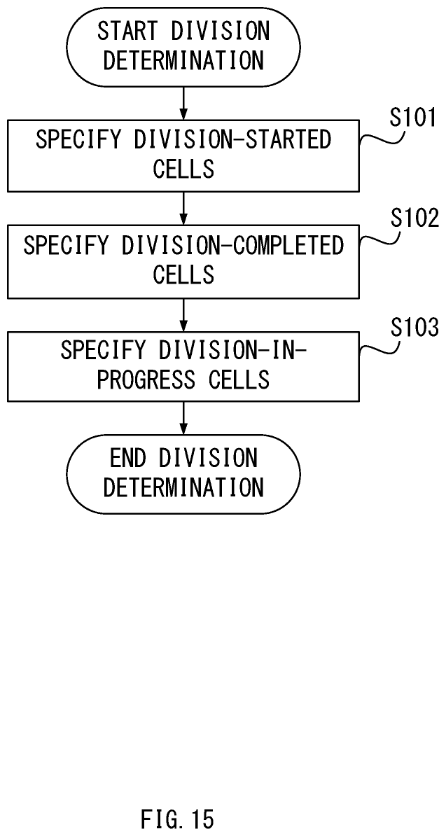

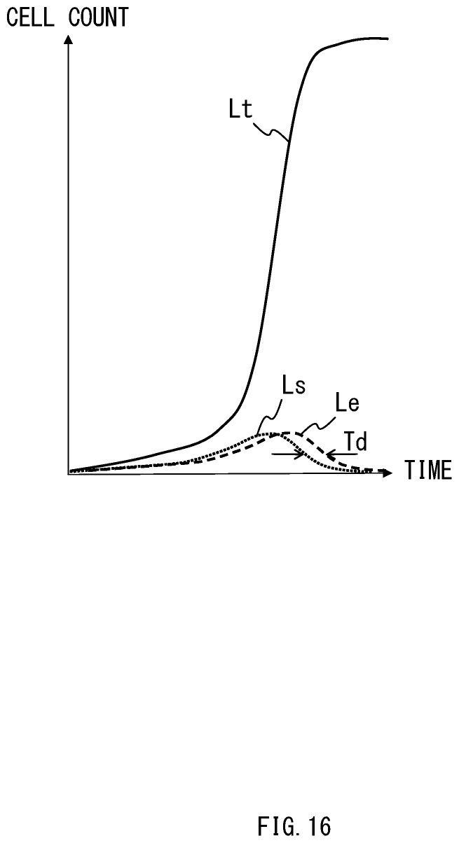

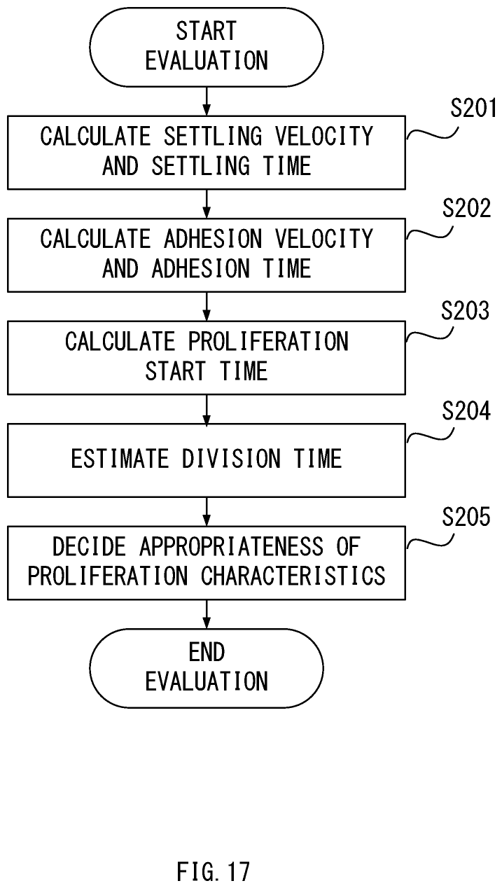

FIG. 14 is a flowchart for processes in accordance with the present embodiment. FIG. 15 is a flowchart for the division determination process in accordance with the present embodiment. FIG. 16 is an example of a graph indicating temporal changes in a total cell count, a division-started cell count, and a division-completed cell count. FIG. 17 is a flowchart for the evaluation process in accordance with the present embodiment. By referring to FIGS. 14-17, the following specifically describes a method for evaluating an adherent cell that is implemented by the evaluation system.

As indicated in FIG. 14, the method for evaluating an adherent cell that is implemented by the evaluation system includes an image obtainment process (step S10), an adhesion determination process (step S20), a division determination process (step S100), a counting process (step S30), an evaluation process (step S200), and a display process (step S300). The image obtainment process, the adhesion determination process, and the counting process are similar to those described above with reference to the first embodiment, and detailed descriptions thereof are omitted herein.

The evaluation system performs the division determination process (step S100), after obtaining an image of adherent cells in the image obtainment process in step S10 and determining whether the adherent cells are adhering in the adhesion determination process in step S20. In step S100, the evaluation system specifies a division state of the adherent cells on the basis of the adhesion determination result obtained in step S20. In step S100, the division determination process is performed for each of the plurality of cells C seen in the image. The following descriptions are given for an exemplary situation in which a N-th image capturing operation is currently performed in time-lapse image capturing.

In step S100, for example, the evaluation system may perform the division determination process depicted in FIG. 15. In the division determination process depicted in FIG. 15, the evaluation system first specifies cells that have started to divide (hereinafter, “division-started cells”) (step S101). In step S101, on the basis of the result of the adhesion determination process in step S20, the control apparatus 30 specifies, as division-started cells, cells that have changed from the adhering state to the non-adhering state. In particular, the control apparatus 30 first obtains coordinates Ga(N−1) of cells specified as adhering cells in an image obtained in the (N−1)-th image capturing operation. Then, the control apparatus 30 obtains coordinates Ga(N) of cells specified as non-adhering cells located at the coordinates Ga(N−1) or in the vicinity thereof in an image obtained in the N-th image capturing operation. Finally, the control apparatus 30 specifies, as division-started cells, the cells located at the coordinates Gb(N) in the image obtained in the N-th image capturing operation and calculates the number of coordinates Gb(N) as a division-started cell count NS(N) for cells that started to divide in the period from the (N−1)-th image capturing operation to the N-th image capturing operation.

Next, the evaluation system specifies cells that have completely divided (hereinafter, “division-completed cells”) (step S102). In step S102, the control apparatus 30 specifies, as division-completed cells, cells that are each a result of a division-started cell specified in step S101 changing into a pair of cells located close to each other. In addition, the evaluation system specifies cells in the process of dividing (hereinafter, “division-in-progress cells”) (step S103) and ends the division determination process. In step S103, the control apparatus 30 specifies, as division-in-progress cells, cells that have not completely divided yet among the division-started cells specified in step S101.

In particular, the control apparatus 30 first obtains coordinates Gb(N−1) of cells specified as division-started cells in an image obtained in the (N−1)-th image capturing operation. Then, the control apparatus 30 obtains coordinates Gc(N) of cells specified as non-adhering cells located at the coordinates Gb(N−1) or in the vicinity thereof in an image obtained in the N-th image capturing operation. In addition, the control apparatus 30 obtains, in the image obtained in the N-th image capturing operation, coordinates Gcp(N) among the coordinates Gc (N) that are distant from other coordinates Gc (N) by a distance less than a threshold, and obtains the other coordinates Gcc(N) (i.e., the coordinates among the coordinates Gc(N) that are distant from other coordinates Gc(N) by a distance greater than the threshold). Finally, the control apparatus 30 specifies, as division-completed cells, the cells located at the coordinates Gcp (N) in the image obtained in the N-th image capturing operation and calculates ½ of the number of coordinates Gcp (N) as a division-completed cell count NE (N) for cells that completely divided in the period from the (N−1)-th image capturing operation to the N-th image capturing operation. The control apparatus 30 also specifies, as division-in-progress cells, the cells located at the coordinates Gcc(N) in the image obtained in the N-th image capturing operation and calculates the number of coordinates Gcc(N) as a division-in-progress cell count Nc(N) for cells that were in the process of dividing in the period from the (N−1)-th image capturing operation to the N-th image capturing operation.

In time-lapse image capturing, the control apparatus 30 may obtain the information depicted in FIG. 16 by repeatedly performing the processes of steps S10, S20, S100, and S30. In FIG. 16, the horizontal axis indicates the elapsed time since seeding, and the vertical axis indicates the number of cells specified from images. A solid line Lt indicates a change in the total cell count specified from the images, a dotted line Ls indicates a temporal change in the division-started cell count specified in the division determination process, and a dashed-line Le indicates a temporal change in the division-completed cell count specified in the division determination process. FIG. 16 indicates a situation in which an increase in the division-completed cell count delays in comparison with an increase in the division-started cell count. For example, a time Td of the delay may be calculated to estimate a division time, i.e., a time required for the division.

Next, the evaluation system performs the evaluation process (step S200). In step S200, the evaluation system evaluates various characteristics of the cells C on the basis of the determination results obtained in steps S20 and S100. In particular, in step S200, the evaluation system performs the evaluation process depicted in FIG. 17.

In the evaluation processing depicted in FIG. 17, first, the evaluation system calculates a settling velocity and a settling time (step S201), calculates an adhesion velocity and an adhesion time (step S202), and calculates a proliferation start time (step S203). The processes of steps S201-S203 are similar to those of steps S31-S33 depicted in FIG. 12. Accordingly, detailed descriptions of these processes are omitted herein.

Next, the evaluation system estimates a division time (step S204). In step S204, the control apparatus 30 estimates a division time extending from the start to end of division on the basis of a temporal change in the division-started cell count and a temporal change in the division-completed cell count. In particular, for example, as indicated in FIG. 16, the time difference between the time at which the division-started cell count becomes half of a peak after reaching the peak and the time at which the division-completed cell count becomes half of a peak after reaching the peak may be estimated as a division time. Alternatively, the time difference between the time at which the division-started cell count reaches a peak and the time at which the division-completed cell count reaches a peak may be estimated as a division time.

Finally, the evaluation system decides the appropriateness of the proliferation characteristics of the cells C (step S205) and ends the evaluation process. In step S205, first, the control apparatus 30 compares the division-completed cell count with an increase in the total cell count that was attained during image capturing and determines whether the line Lt indicated in FIG. 16 is reflecting the proliferation characteristics. When the division-completed cell count is largely different from the increase in the total cell count, the characteristics indicated by the line Lt in FIG. 16 is dominantly affected by the inflow and outflow of cells to/from an observation field of view, and it is determined that the proliferation characteristics are not properly represented. When the difference between the division-completed cell count and the increase in the total cell count is sufficiently small, it is decided that the characteristics indicated by the line Lt in FIG. 16 reflect the proliferation characteristics of the cells C.

Upon finishing the evaluation process, the evaluation system, at a request from a terminal apparatus, outputs various information to the terminal apparatus and causes the display unit of the terminal apparatus to display the information (step S300). In this case, the control apparatus 30 may cause the display unit of the terminal apparatus to display the adhesion determination result obtained in step S20 in, for example, the graph form depicted in FIG. 11. The control apparatus 30 may also cause the display unit of the terminal apparatus to display the division determination result obtained in step S100 in, for example, the graph form depicted in FIG. 16. Furthermore, the control apparatus 30 may cause the display unit of the terminal apparatus to display the evaluation result obtained in step S200 in, for example, a table form.

The evaluation system configured as described above determines whether an adherent cell is adhering on the basis of the profile shape of a contact portion. Accordingly, as with the evaluation system 1, the evaluation system in accordance with the present embodiment can accurately perform the adhesion determination for an adherent cell irrespective of the cell type. The evaluation system in accordance with the present embodiment determines whether an adherent cell has been dividing on the basis of an adhesion determination result. Hence, an increase in a cell count that occurs in association with division can be directly detected, unlike in a situation in which an increase in a cell count that occurs in association with division is indirectly detected according to a change in the cell count. In this way, accurate information pertaining to division can be obtained so that the proliferation characteristics of cells can be correctly evaluated.

The evaluation system can identify the state of cells in a division process in detailed categories, e.g., cells that have just started to divide (division-started cells), cells that have completely divided (division-completed cells), cells in the process of dividing (division-in-progress cells). Displaying these cells in a graph form as depicted in FIG. 16 allows the condition of proliferation caused by division to be intuitively grasped. The number of cells is individually counted for each different cell state, so that a new indicator such as division time that cannot be obtained by performing evaluation using the prior art can be obtained. Accordingly, the state of cell culturing can be grasped in a more detailed manner.

### Third Embodiment

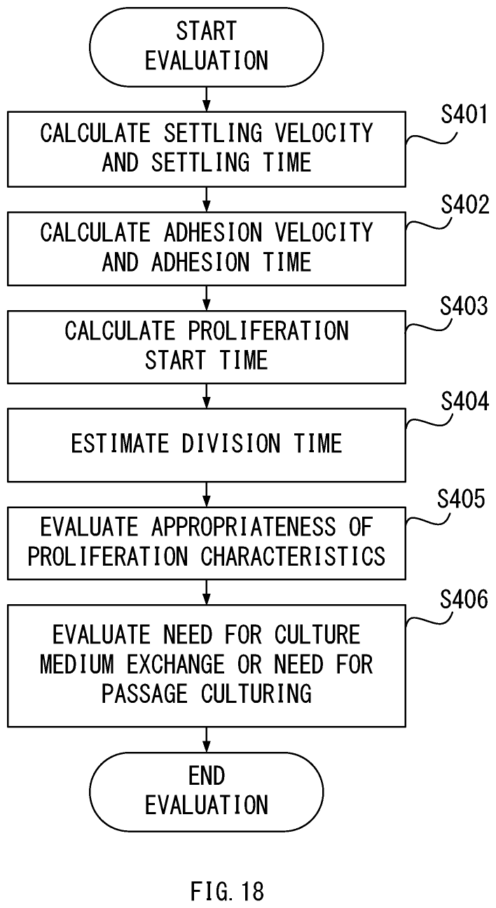

FIG. 18 is a flowchart for an evaluation process in accordance with the present embodiment. The evaluation system in accordance with the present embodiment is different from the evaluation system in accordance with the second embodiment in that the former evaluation system performs the evaluation process indicated in FIG. 18, instead of the evaluation process indicated in FIG. 17. By referring to FIG. 18, the following specifically describes the evaluation process performed by the evaluation system in accordance with the present embodiment (hereinafter simply referred to as the “evaluation system”).

Upon starting the evaluation process indicated in FIG. 18, the evaluation system calculates a settling velocity and a settling time (step S401), calculates an adhesion velocity and an adhesion time (step S402), calculates a proliferation start time (step S403), estimates a division time (step S404), and evaluates the appropriateness of proliferation characteristics (step S405). The processes of steps S401-S405 are similar to those of steps S201-S205 depicted in FIG. 17. Accordingly, detailed descriptions of these processes are omitted herein.

Then, the evaluation system evaluates the need for culture medium exchange or the need for passage culturing (subculturing). In step S406, the control apparatus 30 estimates a period in which the culture medium should be exchanged or passage culturing should be performed on the basis of the division state of adherent cells that is obtained through the division determination process. In particular, for example, the control apparatus 30 may determine whether a division-started cell count or a division-completed cell count has increased in a proliferation period. When determining that neither the division-started cell count nor the division-completed cell count has increased, the control apparatus 30 may determine that the culture medium needs to be exchanged, and report the need to exchange the culture medium in the display process to be performed later. The control apparatus 30 may monitor the division-started cell count or the division-completed cell count after the culture medium exchange and report the effect of the culture medium exchange. For example, when neither the division-started cell count nor the division-completed cell count increases even after replacing the culture medium, the control apparatus 30 may determine that subculturing needs to be performed, and report the need to perform subculturing in the display process to be performed later. Furthermore, the control apparatus 30 may determine whether the division-started cell count or the division-completed cell count has fallen below a threshold. When determining that the division-started cell count or the division-completed cell count has fallen below a threshold, the control apparatus 30 may determine that subculturing needs to be performed, and report the need to perform subculturing in the display process to be performed later.

As with the evaluation system 1, the evaluation system in accordance with the present embodiment can accurately perform the adhesion determination for adherent cells irrespective of the cell type. As with the evaluation system in accordance with the second embodiment, the evaluation system in accordance with the present embodiment can correctly evaluate the proliferation characteristics of cells.

In addition, the evaluation system can determine whether a culture medium needs to be replaced or whether subculturing needs to be performed, on the basis of quantitative information pertaining to the proliferation state of cells obtained from a division determination result. Thus, necessary tasks can be performed at more appropriate timings than in the prior art in which decisions are made on the basis of a person's instincts or experiment (e.g., the deciding of a culture medium replacement timing based on the color of a culture medium, the deciding of a subculturing timing based on a human deciding a confluent state according to an image).

The embodiments described above indicate specific examples to facilitate understanding of the invention, and the present invention is not limited to these embodiments. Some of the embodiments described above may be applied to other embodiments. Various modifications or changes can be made to the method for evaluating an adherent cell, the program, the recording medium, and the system for evaluating an adherent cell without departing from the recitation in the claims.

In the examples indicated for the embodiments described above, it is determined whether an adhering state is achieved in the adhesion determination process on the basis of the profile shape of a contact portion specified by the control apparatus 30. However, the control apparatus 30 may first determine the presence/absence of a pseudopod of an adherent cell on the basis of the profile shape of a specified contact portion. For example, the presence/absence of a pseudopod may be determined on the basis of a roundness calculated from the specified contact portion. Alternatively, a learned model may be created using teacher data for which the presence/absence of a pseudopod is known, and the presence/absence of a pseudopod may be determined using the learned model. When it is determined that there is a pseudopod, it may be decided that the adherent cell is adhering to the culture container.

In the example indicated for the embodiments described above, a time at which the number of adhering cells reaches a certain threshold is specified as a proliferation start time. However, the method for specifying a proliferation start time is not limited to this example. A time at which a non-adhering cell count that has been decreasing starts to increase may be specified as a proliferation start time. This is because the state in which non-adhering cells that have been decreasing in number starts to increase suggests that a change in the cells from the adhering state to the non-adhering state has been progressing because of division.

While descriptions have been given mainly to the processes performed by the evaluation system 1, the following describes the flow of tasks performed when the user evaluates cultured cells by using the system 1. Upon setting the culture container 100 on the image capturing apparatus 10 set within the incubator 20, the user instructs the evaluation system 1 to start image capturing. Upon receipt of the instruction from the user, the evaluation system 1 starts the processes indicated in FIG. 8 or FIG. 14, thereby performing the image obtainment process, the adhesion determination process, the division determination process, the counting process, the evaluation process, and the display process. The timing at which the image obtainment process is performed may be synchronous with the timings at which the other processes are performed, or the image obtainment process may be performed independently of the other processes. Thus, every time an image is obtained, the determination process, the counting process, the evaluation process, and the display process may be performed. Alternatively, after images are obtained and a certain number of images are accumulated, the determination process, the counting process, the evaluation process, and the display process may be performed. In another example, after all the images are obtained, the determination process, the counting process, the evaluation process, and the display process may be performed. Afterward, the user evaluates cultured cells by checking information displayed in the display process. As described above, the user can evaluate cultured cells on the basis of information provided from the evaluation system 1. Accordingly, the evaluation system 1 can assist the user evaluating cultured cells, in addition to evaluating cultured cells by itself.

What is claimed is:

1. An adherent-cell evaluation method comprising:
obtaining an image of an adherent cell accommodated within a culture container; and
determining whether the adherent cell is adhering to the culture container based on at least a profile shape of a contact portion of the adherent cell that is in contact with the culture container, the profile shape being specified from the image.

2. The evaluation method of claim 1, wherein
the determining whether the adherent cell is adhering to the culture container includes
specifying the profile shape of the contact portion based on the image,
determining a presence/absence of a pseudopod of the adherent cell based on the specified profile shape of the contact portion, and
determining that the adherent cell is adhering to the culture container when determining that a pseudopod is present.

3. The evaluation method of claim 1, wherein
the determining whether the adherent cell is adhering to the culture container includes
specifying the profile shape of the contact portion based on the image, and
determining whether the adherent cell is adhering to the culture container based on a deviation of the specified profile shape of the contact portion from a standard shape.

4. The evaluation method of claim 1, wherein
the determining whether the adherent cell is adhering to the culture container includes
specifying the profile shape of the contact portion based on the image,
determining a presence/absence of irregularities on a surface of the adherent cell based on the image, and
determining whether the adherent cell is adhering to the culture container based on at least the profile shape of the contact portion and the presence/absence of the irregularities.

5. The evaluation method of claim 1, wherein
the determining whether the adherent cell is adhering to the culture container includes
specifying the profile shape of the contact portion based on the image,
determining a presence/absence of irregularities on a surface of the adherent cell based on the image,
determining, based on the image, whether a size of the adherent cell for which it has been determined that irregularities are present is equal to or greater than a threshold, and
determining whether the adherent cell is adhering to the culture container based on at least the profile shape of the contact portion, the presence/absence of irregularities, and whether the size of the adherent cell is equal to or greater than the threshold.

6. The evaluation method of claim 2, wherein
the determining whether the adherent cell is adhering to the culture container includes
specifying the profile shape of the contact portion based on the image,
determining a presence/absence of irregularities on a surface of the adherent cell based on the image,
determining, based on the image, whether a size of the adherent cell for which it has been determined that irregularities are present is equal to or greater than a threshold, and
determining whether the adherent cell is adhering to the culture container based on at least the profile shape of the contact portion, the presence/absence of irregularities, and whether the size of the adherent cell is equal to or greater than the threshold.

7. The evaluation method of claim 3, wherein
the determining whether the adherent cell is adhering to the culture container includes
specifying the profile shape of the contact portion based on the image,
determining a presence/absence of irregularities on a surface of the adherent cell based on the image,
determining, based on the image, whether a size of the adherent cell for which it has been determined that irregularities are present is equal to or greater than a threshold, and
determining whether the adherent cell is adhering to the culture container based on at least the profile shape of the contact portion, the presence/absence of irregularities, and whether the size of the adherent cell is equal to or greater than the threshold.

8. The evaluation method of claim 1, further comprising:
specifying a division state of the adherent cell based on a result of the determining whether the adherent cell is adhering to the culture container.

9. The evaluation method of claim 2, further comprising:
specifying a division state of the adherent cell based on a result of the determining whether the adherent cell is adhering to the culture container.

10. The evaluation method of claim 3, further comprising:
specifying a division state of the adherent cell based on a result of the determining whether the adherent cell is adhering to the culture container.

11. The evaluation method of claim 4, further comprising:
specifying a division state of the adherent cell based on a result of the determining whether the adherent cell is adhering to the culture container.

12. The evaluation method of claim 5, further comprising:
specifying a division state of the adherent cell based on a result of the determining whether the adherent cell is adhering to the culture container.

13. The evaluation method of claim 6, further comprising:
specifying a division state of the adherent cell based on a result of the determining whether the adherent cell is adhering to the culture container.

14. The evaluation method of claim 8, wherein
the specifying the division state of the adherent cell includes specifying, as a cell that has started to divide, an adherent cell that has changed from an adhering state to a non-adhering state.

15. The evaluation method of claim 14, wherein
the specifying the division state of the adherent cell further includes
specifying, as cells that have completely divided, a pair of cells located close to each other that have been changed from a cell that started to divide, and
the evaluation method further comprises estimating a time extending from a start to end of division based on a temporal change in a number of cells that have started to divide and a temporal change in a number of cells that have completely divided.

16. The evaluation method of claim 8, further comprising:
estimating, based on the division state of the adherent cell, a period to be exchange a culture medium or to be performed subculturing.

17. The evaluation method of claim 1, wherein
the obtaining an image includes
obtaining a plurality of images for different focus positions,
calculating contrasts of the plurality of images, and
selecting, from the plurality of images based on the contrasts of the plurality of images, a selection image to be used in the determining whether the adherent cell is adhering to the culture container, and
the determining whether the adherent cell is adhering to the culture container includes determining, based on the selection image, whether the adherent cell is adhering to the culture container.

18. A non-transitory computer-readable medium having recorded therein a program for causing a computer to perform a process comprising
obtaining an image of an adherent cell accommodated within a culture container; and
determining whether the adherent cell is adhering to the culture container based on at least a profile shape of a contact portion of the adherent cell that is in contact with the culture container, the profile shape being specified from the image.

19. An evaluation system for evaluating an adherent cell, the evaluation system comprising:
an image capturing apparatus that captures an image of an adherent cell accommodated within a culture container; and
a processor, wherein
the processor
obtains the image obtained by the image capturing apparatus, and
determines whether the adherent cell is adhering to the culture container based on at least a profile shape of a contact portion of the adherent cell that is in contact with the culture container, the profile shape being specified from the image.

20. The evaluation system of claim 19, wherein
the image capturing apparatus includes
a stage on which the culture container is placed,
an image capturing element disposed below the stage, and
a light source that is disposed around the image capturing element and illuminates the culture container from below the stage.
